(19) United States
(12) Patent Application Publication
(10) Pub. No.: US 2019/0185890 A1
(43) Pub. Date: Jun. 20, 2019

(54) METHOD FOR THE FERMENTATIVE PRODUCTION OF L-LYSINE

(71) Applicant: Evonik Degussa GmbH, Essen (DE)

(72) Inventors: Kornelia VOSS, Steinhagen (DE); Georg THIERBACH, Bielefeld (DE); Thomas BEKEL, Halle (Westf.) (DE)

(21) Appl. No.: 16/219,718

(22) Filed: Dec. 13, 2018

(30) Foreign Application Priority Data
Dec. 14, 2017 (EP) 17207318.1

(51) Int. Cl. C12P 13/08; C12N 15/77; C12N 15/10; C12R 1/15

(52) U.S. Cl. CPC C12P 13/08; C12R 1/15; C12N 15/1024; C12N 15/77

(57) ABSTRACT

The present invention makes available novel L-lysine excreting bacteria of the species Corynebacterium glutamicum, having the ability to excrete L-lysine, containing in their chromosome a polynucleotide encoding a polypeptide having the activity of a malate:quinone reductase wherein the amino acid at position 228 of the amino acid sequence of the polypeptide contains any proteinogenic amino acid different from valine and a method for producing L-lysine using such bacteria.

### CROSS REFERENCE TO RELATED APPLICATIONS

The present application claims priority under 35 USC § 119 to European application, EP 17207318.1, filed on Dec. 14, 2017, the contents of which is incorporated herein by reference in its entirety.

### FIELD OF THE INVENTION

The invention relates to the fermentative production of amino acids and bacteria that can be used for this purpose.

### BACKGROUND OF THE INVENTION

L-lysine is used in human medicine, in the pharmaceutical industry, in the food industry and particularly in nutrition of animals.

It is known that L-lysine is produced by fermentation of strains of the species Corynebacterium glutamicum. Because of the great economic importance, work is continually being done on improving the production methods. Improvements may relate to the fermentation technology such as e. g. stirring and supplying oxygen, or to the composition of the nutrient media e.g. the sugar concentration during fermentation, or to the processing of the fermentation broth to a suitable product form by e. g. drying and granulating the fermentation broth or ion exchange chromatography or may relate to the intrinsic performance properties of the microorganism itself.

The methods used for improving the performance properties of these microorganisms are those of mutagenesis, selection and screening of mutants. The strains obtained in this way are resistant to anti-metabolites or are auxotrophic for metabolites of regulatory importance, and produce L-lysine. A well-known anti-metabolite is the L-lysine analogue S-(2-aminoethyl)-L-cysteine (see e.g., Tosaka, et al.: Agricultural and Biological Chemistry 42(4):745-752, (1978)).

Methods of recombinant DNA technology have likewise been used for a number of years for improvement of L-lysine-producing strains of the species Corynebacterium glutamicum, by modifying, i.e. enhancing or attenuating, individual L-lysine biosynthesis genes and investigating the effect on L-lysine production.

The nucleotide sequences of the chromosomes of various bacteria or strains resp. of the species Corynebacterium glutamicum, and their analysis have been disclosed. This information is available at publicly accessible data bases and may be used for strain development purposes. One such data base is the GenBank data base of the NCBI (National Center for Biotechnology Information, U.S. National Library of Medicine 8600 Rockville Pike, Bethesda Md., 20894 USA).

During the annotation procedure for a sequenced chromosome of an organism identified structures such as e. g. genes or coding sequences are furnished with a unique identifier called locus_tag by the supplier of the information to the data base.

The nucleotide sequence of the Corynebacterium glutamicum ATCC13032 chromosome and its analysis were described by Ikeda and Nakagawa (Applied Microbiology and Biotechnology 62:99-109(2003)) and in EP1108790A2. The information is available at the NCBI under accession number NC_003450. In the chromosome sequence disclosed under accession number NC_003450 locus_tag NCg11926 identifies a nucleotide sequence coding for a malate:quinone oxidoreductase. The amino acid sequence of the polypeptide is available under the identifier NP_60120.

The nucleotide sequence of the Corynebacterium glutamicum ATCC13032 chromosome and its analysis were independently described by Kalinowski, et al. (Journal of Biotechnology 104 (1-3):5-25 (2003)). The information is available at the NCBI under accession number NC_006958. Locus_tag CGTRNA_RS09800 identifies a nucleotide sequence coding for a malate:quinone oxidoreductase. The old_locus_tag designation cg2192 is also used in the art. The amino acid sequence of the polypeptide is available under the identifier WP_011014814.

The nucleotide sequences of locus_tag NCg11926 and CGTRNA_RS09800 are identical.

Lv, et al. (Journal of Bacteriology 194(3):742-743 (2012)) describe the sequencing of the chromosome of Corynebacterium glutamicum ATCC14067, a strain formerly referred to as Brevibacterium flavum. The information is available at the NCBI under accession number AGQQ02000001 and AGQQ02000002. The nucleotide sequence of the chromosome of Corynebacterium glutamicum ATCC13869, a strain formerly referred to as Brevibacterium lactofermentum, and its analysis were disclosed by Chen, et al. at the NCBI under accession number NZ_CP016335.

Malate:quinone oxidoreductase is a membrane associated enzyme catalyzing the oxidation of malate to oxaloacetate with quinones serving as electron acceptors. The biochemical and genetic characterization of the enzyme of Corynebacterium glutamicum and its function were described by Molenaar, et al. (European Journal of Biochem. 254:395-403 (1998)) and Molenaar, et al. (Journal of Bacteriology 182(24):6884-6891 (2000)). Molenaar, et al. referred to the enzyme as EC 1.1.99.16. According to current IUBMB Enzyme Nomenclature the enzyme is given the systematic designation EC 1.1.5.4 with malate dehydrogenase (quinone) as accepted name and (S)-malate:quinone oxidoreductase as systematic name. The art uses the abbreviation Mqo for the enzyme or for the polypeptide resp. The gene encoding the Mqo polypeptide is called mqo. Information concerning transcription signals, e.g., −10 region of a promoter, the 5′-untranslated leader sequence (5′-UTR), and translation signals, e.g., ribosome binding site (RBS), of the mqo gene (old_locus_tag cg2192) can be found in Pfeifer-Sancar, et al. (BMC Genomics 14:888 (2013)) and Han, et al. (Journal of Molecular Microbiology and Biotechnology 15:264-276, 2008).

US20020028490A1 describes the favorable effect of overexpression of malate:quinone oxidoreductase on L-lysine and L-threonine production by Corynebacterium glutamicum.

WO02086137A2 describes the favorable effect of attenuating or eliminating the malate:quinone oxidoreductase activity on L-lysine production by Corynebacterium glutamicum.

WO03029457A1 likewise describes the favorable effect of attenuating or eliminating the malate:quinone oxidoreductase activity on L-lysine production by Corynebacterium glutamicum.

US20060166338A1 describes the favorable effect of an amino acid exchange at position 111 of the amino acid sequence of the malate:quinone oxidoreductase on L-lysine production in Corynebacterium glutamicum.

### DESCRIPTION OF THE INVENTION

Object of the present invention is to provide new measures for the fermentative production of L-lysine by bacteria of the species Corynebacterium glutamicum.

To achieve the object outlined above the present invention makes available novel L-lysine excreting bacteria of the species Corynebacterium glutamicum, having the ability to excrete L-lysine, containing in their chromosome a polynucleotide encoding a polypeptide having the activity of a malate:quinone reductase wherein the amino acid at position 228 of the amino acid sequence of the polypeptide contains any proteinogenic amino acid different from valine.

The present invention further makes available methods for producing said L-lysine by using said bacteria in a fermentative process and for the manufacturing of a product containing said L-lysine from the fermentation broth.

The objects underlying the present invention were solved by the means as written in the claims.

Accordingly the present invention provides the following:

A bacterium of the species Corynebacterium glutamicum, having the ability to excrete L-lysine, containing in its chromosome a polynucleotide encoding a polypeptide having the activity of a malate:quinone oxidoreductase and comprising the amino acid sequence of SEQ ID NO:2, wherein the amino acid valine at position 228 is substituted by a different proteinogenic amino acid, preferably by aspartic acid or glutamic acid, particular preferred by aspartic acid.

It was found that the bacteria modified according to the invention excreted L-lysine, into a suitable medium under suitable fermentation conditions in an increased manner with respect to one or more parameters selected from product concentration (i.e., amount of L-lysine produced per volume, or mass unit resp., of medium/fermentation broth (e.g. g/l or g/kg), product yield (i.e., amount of L-lysine produced per carbon source consumed (e.g., g/g or kg/kg)), product formation rate (i.e., amount of L-lysine produced per volume, or mass unit resp., of medium/fermentation broth and unit of time (e.g., g/l×h or g/kg×h)), and specific product formation rate (i.e., amount of L-lysine produced per unit of time and mass unit of the producer (e.g., g/h×g dry mass)) as compared to the unmodified bacterium.

It is obvious that a higher product concentration facilitates product manufacturing e. g. purification and isolation. An increased product yield reduces the amount of raw material required. An increased product formation rate reduces the time required for a fermentation run thus increasing the availability of a given fermenter.

The bacteria according to the invention thus contribute to the improvement of technical and economic aspects of the manufacturing of L-lysine or L-lysine containing products.

In a further embodiment the bacterium according to the invention contains in its chromosome a polynucleotide encoding an amino acid sequence of a polypeptide having malate:quinone oxidoreductase activity comprising the amino acid sequence according to SEQ ID NO:2, wherein the amino acid valine at position 228 of the encoded amino acid sequence of SEQ ID NO:2 is substituted by aspartic acid, and comprising the nucleotide sequence of positions 1001 to 2500 of SEQ ID NO:1 the nucleobase at position 1683 being adenine or comprising the nucleotide sequence of positions 1001 to 2500 of SEQ ID NO:3.

In a further embodiment the bacterium according to the invention contains in its chromosome a polynucleotide which is coding for an amino acid sequence of a polypeptide having malate:quinone oxidoreductase activity comprising the amino acid sequence according to SEQ ID NO:2, wherein the amino acid valine at position 228 of the encoded amino acid sequence of SEQ ID NO:2 is substituted by a different proteinogenic amino acid, preferably by aspartic acid or glutamic acid, particularly preferred by aspartic acid, and wherein the 5′-end of the coding sequence of this polynucleotide is directly connected to the 3′-end of the polynucleotide comprising the nucleotide sequence from positions 781 to 1000 of SEQ ID NO:1 or of SEQ ID NO:3. In particular, the bacterium of the species Corynebacterium glutamicum according to the present invention contains in its chromosome a polynucleotide encoding the polypeptide comprising the amino acid sequence according to SEQ ID NO:2 the amino acid at position 228 being aspartic acid and having the activity of a malate: quinone oxidoreductase which comprises the nucleotide sequence from positions 781 to 2500 of SEQ ID NO:3 or the nucleotide sequence from positions 781 to 2500 of SEQ ID NO:1 the nucleobase at position 1683 being adenine. In other words, the bacterium according to the present invention may contain in its chromosome a polynucleotide encoding said amino acid sequence which comprises the nucleotide sequence from positions 781 to 2500 of SEQ ID NO:1 whereby the nucleobase at position 1683 is adenine or the nucleotide sequence from positions 781 to 2500 of SEQ ID NO:3.

The term L-lysine, where mentioned herein, in particular in the context of product formation, also comprises their ionic forms and salts, for example L-lysine mono hydrochloride or L-lysine sulfate.

For practicing the present invention bacteria of the species Corynebacterium glutamicum are used. A description of the genus Corynebacterium and the species Corynebacterium glutamicum comprised by this genus can be found in the article “Corynebacterium” by K. A. Bernard and G. Funke in Bergey's Manual of Systematics of Archaea and Bacteria (Bergey's Manual Trust, 2012).

Suitable strains of Corynebacterium glutamicum are wild strains of this species for example strains ATCC13032, ATCC14067 and ATCC13869, and L-lysine excreting strains obtained from these wild strains.

Strain ATCC13032 (also available as DSM20300) is the taxonomic type strain of the species Corynebacterium glutamicum. Strain ATCC14067 (also available as DSM20411) is also known under the outdated designation Brevibacterium flavum. Strain ATCC13869 (also available as DSM1412) is also known under the outdated designation Brevibacterium lactofermentum. A taxonomic study of this group of bacteria based on DNA-DNA hybridization was done by Liebl, et al. (International Journal of Systematic Bacteriology 41(2):255-260, 1991). A comparative analysis of various strains of the species Corynebacterium glutamicum based on genome sequence analysis was provided by Yang and Yang (BMC Genomics 18(4940).

A multitude of L-lysine excreting strains of the genus Corynebacterium, in particular of the species Corynebacterium glutamicum were obtained in the art during the past decades starting from strains such as ATCC13032, ATCC14067, ATCC13869 and the like. They were obtained as a result of strain development programs using inter alia methods like classical mutagenesis, selection for antimetabolite resistance as well as amplification and promotor modification of genes of the biosynthetic pathway of the L-amino acid in question by genetic engineering methods. Summaries may be found in L. Eggeling and M. Bott (Handbook of Corynebacterium glutamicum, CRC Press, 2005) or H. Yukawa and M. Inui (Corynebacterium glutamicum Biology and Biotechnology, Springer Verlag, 2013).

L-lysine excreting strains of the species Corynebacterium glutamicum are widely known in the art and can be used for the purpose of the present invention. For example Blombach, et al. (Applied and Environmental Microbiology 75(2):419-427, 2009) describe strain DM1933, which is deposited under accession DSM25442 according to the Budapest treaty. Strain DM1933 was obtained from ATCC13032 by several steps of strain development. Furthermore L-lysine excreting Corynebacterium glutamicum strain DM2031, deposited according to the Budapest Treaty as DSM32514 may be used. Strain DM2031 is a further developed derivative of DM1933 having enhanced L-lysine excretion ability. Other L-lysine excreting Corynebacterium glutamicum strains are, e.g., described in WO 2008033001A1 and EP 0841395A1.

L-lysine excreting strains of the species Corynebacterium glutamicum typically contain a polynucleotide coding for a feedback resistant aspartokinase polypeptide variant. A feedback resistant aspartokinase polypeptide variant means an aspartokinase which is less sensitive, or desensitized resp., to inhibition by mixtures of L-lysine and L-threonine, e.g., 10 mM each, or mixtures of the L-lysine analogue S-(2-aminoethyl)-L-cysteine and L-threonine, e.g., 50 mM S-(2-aminoethyl)-L-cysteine and 10 mM L-threonine, when compared to the wild form of the enzyme, which is contained in wild strains like for example ATCC13032, ATCC14067 and ATCC13869. The EC number for aspartokinase is EC 2.7.2.4. Descriptions of polynucleotides of Corynebacterium glutamicum encoding a feedback resistant aspartokinase polypeptide variant are for example given in U.S. Pat. Nos. 5,688,671, 6,844,176 and 6,893,848. A summarizing list can be found inter alia in US 2009/0311758 A1. The symbol used in the art for a gene coding for an aspartokinase polypeptide is lysC. In case the gene codes for a feedback resistant polypeptide variant the art typically uses symbols like lysC^{fbr} with fbr indicating feedback resistance.

Accordingly said L-lysine excreting strains of the species Corynebacterium glutamicum used for the measures of the present invention preferably contain at least one copy of a polynucleotide coding for a feedback resistant aspartokinase polypeptide.

SEQ ID NO:5 shows the nucleotide sequence of the coding sequence of the aspartokinase polypeptide of strain ATCC13032 and SEQ ID NO:6 the amino acid sequence of the encoded polypeptide. It is known in the art (see U.S. Pat. No. 6,893,848) that exchange of the amino acid Thr at position 311 of SEQ ID NO:6 for Ile imparts the enzyme feedback resistance to inhibition by mixtures of L-lysine and L-threonine.

Accordingly it is preferred that the amino acid sequence of said feedback resistant aspartokinase polypeptide comprises the amino acid sequence of SEQ ID NO:6 containing isoleucine at position 311.

Said amino exchange can be achieved by exchanging the nucleobase cytosine (c) at position 932 of SEQ ID NO:5 to give thymine (t). The acc codon for threonine is thus altered to the atc codon for isoleucine.

It is further known in the art that exchange of the gtg start codon of the coding sequence for the aspartokinase polypeptide for atg enhances expression of the polypeptide (see e.g., EP 2796555 A2).

Accordingly it is preferred that the sequence coding for a feedback resistant aspartokinase polypeptide begins with an atg start codon.

Summaries concerning the breeding of L-lysine excreting strains of Corynebacterium glutamicum may be found inter alia in L. Eggeling and M. Bott (Handbook of Corynebacterium glutamicum, CRC Press, 2005), V. F. Wendisch (Amino Acid Biosynthesis—Pathways, Regulation and Metabolic Engineering, Springer Verlag, 2007), H. Yukawa and M. Inui (Corynebacterium glutamicum Biology and Biotechnolgy, Springer Verlag, 2013) or A. Yokota and M. Ikeda (Amino Acid Fermentation, Springer Verlag, 2017), and Eggeling and Bott (Applied Microbiology and Biotechnology 99 (9):3387-3394, 2015).

In case a wild strain, e.g., ATCC13032, ATCC13869 or ATCC14067, is in a first step subjected to the measures of the present invention the resulting strain is in a second step subjected to a strain development program targeted at L-lysine in order to obtain a bacterium according to the present invention.

The term DSM denotes the depository Deutsche Sammlung für Mikroorganismen and Zellkulturen located in Braunschweig, Germany. The term ATCC denotes the depository American Type Culture Collection located in Manasass, Virginia, US.

Details regarding the biochemistry and chemical structure of polynucleotides and polypeptides as present in living things such as bacteria like Corynebacterium glutamicum or Escherichia coli, for example, can be found inter alia in the text book “Biochemie” by Berg et al. (Spektrum Akademischer Verlag Heidelberg, Berlin, Germany, 2003; ISBN 3-8274-1303-6).

Polynucleotides consisting of deoxyribonucleotide monomers containing the nucleobases or bases resp. adenine (a), guanine (g), cytosine (c) and thymine (t) are referred to as deoxyribopolynucleotides or deoxyribonucleic acid (DNA). Polynucleotides consisting of ribonucleotide monomers containing the nucleobases or bases resp. adenine (a), guanine (g), cytosine (c) and uracil (u) are referred to as ribo-polynucleotides or ribonucleic acid (RNA). The monomers in said polynucleotides are covalently linked to one another by a 3′,5′-phosphodiester bond. By convention single strand polynucleotides are written from 5′- to 3′-direction. Accordingly a polynucleotide has a 5′-end and 3′-end. The order of the nucleotide monomers in the polynucleotide is commonly referred to as nucleotide sequence. Accordingly a polynucleotide is characterized by its nucleotide sequence. For the purpose of this invention deoxyribopolynucleotides are preferred. In bacteria, for example Corynebacterium glutamicum or Escherichia coli, the DNA is typically present in double stranded form. Accordingly the length of a DNA molecule is typically given in base pairs (bp). The nucleotide sequence coding for a specific polypeptide is called coding sequence (cds). A triplett of nucleotides specifying an amino acid or a stop signal for translation is called a codon. The codons specifying different amino acids are well known in the art.

A gene from a chemical point of view is a polynucleotide, preferably a deoxyribopolynucleotide.

DNA can be synthesized de novo by the phoshoramidite method (McBride and Caruthers: Tetrahedon Letters 24(3):245-248 (1983)). Summaries concerning de novo DNA synthesis may be found in the review article of Kosuri and Chruch (Nature Methods 11(5):499-507 (2014)) or in the article of Graf, et al. in the book “Systems Biology and Synthetic Biology” by P. Fu and S. Panke (John Wiley, US, 2009, pp 411-438).

The term gene refers to a polynucleotide comprising a nucleotide sequence coding for a specific polypeptide (coding sequence) and the adjoining stop codon. In a broader sense the term includes regulatory sequences preceding and following the coding sequence. The preceding sequence is located at the 5′-end of the coding sequence and is also referred to as upstream sequence. A promotor is an example of a regulatory sequence located 5′ to the coding sequence. The sequence following the coding sequence is located at its 3′-end and also referred to as downstream sequence. In the context of the present invention said regulatory sequence, comprising inter alia the promotor and the ribosome binding site, of the mqo gene preceding its coding sequence is located from position 781 to 1000 of SEQ ID NO:1 or SEQ ID NO:3. A transcriptional terminator is an example of a regulatory sequence located 3′ to the coding sequence.

Polypeptides consist of L-amino acid monomers joined by peptide bonds. For abbreviation of L-amino acids the one letter code and three letter code of IUPAC (International Union of Pure and Applied Chemistry) is used. Due to the nature of polypeptide biosynthesis polypeptides have an amino terminal end and a carboxyl terminal end also referred to as N-terminal end and C-terminal end. The order of the L-amino acids or L-amino acid residues resp. in the polypeptide is commonly referred to as amino acid sequence. Polypeptides are also referred to as proteins. Further it is known in the art that the start codon or initiation codon resp. gtg of a coding sequence as well as atg encodes the amino acid methionine. It is known in the art that the N-terminal amino acid methionine of an encoded polypeptide may be removed by an aminopeptidase during or after translation (Jocelyn E. Krebs, Elliott S. Goldstein and Stephan T. Kilpatrick: Lewin's Genes X, Jones and Bartlett Publishers, US, 2011).

Proteinogenic L-amino acids are to be understood to mean the L-amino acids present in natural proteins, that is proteins of microorganisms, plants, animals and humans. Proteinogenic L-amino acids comprise L-aspartic acid, L-asparagine, L-threonine, L-serine, L-glutamic acid, L-glutamine, L-glycine, L-alanine, L-cysteine, L-valine, L-methionine, L-isoleucine, L-leucine, L-tyrosine, L-phenylalanine, L-histidine, L-lysine, L-tryptophan, L-arginine, L-proline and in some cases L-selenocysteine and L-pyrrolysine. It is common in the ass to refer to these proteinogenic L-amino acids without hinting to the L configuration at the α carbon atom of the L-amino acid; e.g. L-valine may simply called valine, L-aspartic acid may simply be called aspartic acid etc.

Teachings and information concerning the handling of and experimental work with polynucleotides may be found inter alia in the handbook of J. Sambrook, et al. (Molecular Cloning, A Laboratory Manual, Cold Spring Harbor Laboratory Press, 1989), the textbook of C. R. Newton and A. Graham (PCR, Spektrum Akademischer Verlag, 1994) and the handbook of D. Rickwood and B. D. Hames (Gel electrophoresis of nucleic acids, a practical approach, IRL Press, 1982).

For sequence analysis of polynucleotides and polypeptides, e.g., sequence alignments the Clustal W program (Larkin, et al.: Clustal W and Clustal X version 2.0. In: Bioinformatics 23:2947-2948 (2007)) or public software such as the CLC Genomics Workbench (Qiagen, Hilden, Germany) or the program MUSCLE provided by the European Bioinformatics Institute (EMBL-EBI, Hinxton, UK) may be used.

Corynebacterium glutamicum, in particular strain ATCC13032 and L-lysine excreting strains obtained therefrom during a strain development program, contain in their chromosome among others a gene encoding a polypeptide having the activity of a malate: quinone oxidoreductase and comprising the amino acid sequence of SEQ ID NO:2. The coding sequence is shown SEQ ID NO:1 positions 1001 to 2500. The coding sequence may contain silent mutations which do not alter the amino acid sequence of the Mqo polypeptide. For example the amino acid Phe at position 410 of the amino acid sequence shown in SEQ ID NO:2 or SEQ ID NO:4 may be specified by the codon ttc or by the codon ttt (see positions 2228 to 2230, in particular position 2230, of SEQ ID NO:1 or SEQ ID NO:3). This context is also known as degeneracy of the genetic code in the art.

During the work for the present invention it was found that modifying L-lysine excreting bacteria of the species Corynebacterium glutamicum by exchanging the amino acid valine at position 228 of the encoded amino acid sequence of the Mqo polypeptide shown in SEQ ID NO:2 for a different proteinogenic, preferably aspartic acid or glutamic acid, particular preferred aspartic acid, increased their ability to excrete L-lysine as compared to the unmodified bacterium. The skilled artisan is aware of a number of methods of mutagenesis how to achieve said modification in the Corynebacterium glutamicum.

A mutant bacterium according to the invention can be obtained by classical in vivo mutagenesis executed with cell populations of strains of Corynebacterium glutamicum using mutagenic substances, e.g., N-methyl-N′-nitro-N-nitrosoguanidine, or ultra violet light. The nucleotide sequence comprising the site of mutagenesis within the mqo gene can be amplified by PCR using primers selected from SEQ ID NO:1 or SEQ ID NO:3. By sequencing the PCR product the desired mutants are identified. Details concerning this approach can be found inter alia in U.S. Pat. No. 7,754,446.

A further method is the CRISPR-Cpf1 assisted genome editing described by Jiang, et al. (Nature Communications 8: 15179 DOI: 10; 1038/ncomms15179).

Another common method of mutating genes of Corynebacterium glutamicum is the method of gene replacement described by Schwarzer and Püler (Bio/Technology 9:84-87 (1991)) and further elaborated by Schäfer, et al. (Gene 145:69-73 (1994)).

Peters-Wendisch, et al. (Microbiology 144:915-927 (1998)) used the gene replacement method to inactivate the pyc gene of Corynebacterium glutamicum encoding pyruvate carboxylase. Schäfer, et al. used the method to incorporate a deletion into the hom-thrB gene region of Corynebacterium glutamicum. In EP 1094111 the method was used to incorporate a deletion into the pck gene of Corynebacterium glutamicum encoding phosphoenol pyruvate carboxykinase. In U.S. Pat. No. 7,585,650 the method was applied to the zwf gene to realize an amino acid exchange at position 321 of the amino acid sequence of the Zwf sub-unit of the glucose 6-phosphate dehydrogenase. In U.S. Pat. No. 7,754,446 the method was applied to the rel gene to realize an amino acid exchange at position 38 of the amino acid sequence of the GTP-pyrophosphate kinase polypeptide.

In the gene replacement method, a mutation, for example, a deletion, insertion or substitution of at least one nucleobase, is provided by an isolated polynucleotide comprising the nucleotide sequence of the gene in question or a part thereof containing the mutation.

In the context of the present invention the nucleotide sequence of the gene in question is the mqo gene.

In the context of the present invention the mutation is a substitution of at least one nucleobase located in the codon specifying the amino acid valine at position 228 of the encoded amino acid sequence (see SEQ ID NO:1 and SEQ ID NO:3) of the Mqo polypeptide.

As a consequence of said mutation the codon specifies a proteinogenic amino acid different from valine, preferably aspartic acid or glutamic acid, particular preferred aspartic acid. The codons specifying aspartic acid are gat or gac. The codon gac is preferred.

The codon for the amino acid at position 228 has the position from 1682 to 1684 in SEQ ID NO:1 or SEQ ID NO:3. The nucleotide sequence from position 1682 to 1684, in particular the nucleotide at position 1683, may also be referred to as site of mutation.

The mutated nucleotide sequence of the gene in question or a part thereof containing the mutation comprises i) a nucleotide sequence at the 5′-end of the site of mutation, which is also referred to as 5′-flanking sequence or upstream sequence in the art, ii) a nucleotide sequence at the 3′-end of the site of mutation, which is also referred to as 3′-flanking sequence or downstream sequence in the art, and iii) the nucleotide sequence of the site of mutation between i) and ii).

Said 5′-flanking sequence and 3′-flanking sequence required for homologous recombination typically have a length of at least 200 bp, at least 400 bp, at least 600 bp or at least 800 bp. The maximum length typically is 1000 bp, 1500 bp or 2000 bp.

An example of a mutated nucleotide sequence in the context of the present invention comprises the nucleotide sequence shown in SEQ ID NO:1 from positions 1083 to 2283 with adenine at position 1683. Another example of a mutated nucleotide sequence in the context of the present invention comprises the nucleotide sequence shown in SEQ ID NO:3 from positions 1083 to 2283.

A further example of a mutated nucleotide sequence in the context of the present invention comprises the nucleotide sequence shown in SEQ ID NO:7. The nucleotide sequence of SEQ ID NO:7 corresponds to SEQ ID NO:3 from positions 882 to 2484. SEQ ID NO:7 contains a part of the coding sequence of the mutated mqo gene. Said part of the coding sequence present in SEQ ID NO:7 encodes the N′-terminal end of the Mqo polypeptide i.e. the amino acids from positions 1 to 494. Said part of the coding sequence lacks the sequence coding for the c-terminal end of the Mqo polypeptide (see SEQ ID NO:8).

The 5′-flanking sequence comprises the nucleotide sequence from positions 1 to 801 of SEQ ID NO:7. The 3′-flanking sequence consists of the nucleotide sequence from positions 803 to 1603 of SEQ ID NO:7. The site of mutation is at position 802.

The mutated nucleotide sequence provided is cloned into a plasmid vector that is not capable of autonomous replication in Corynebacterium glutamicum. Said plasmid vector comprising said mutated nucleotide sequence is subsequently transferred into the desired strain of Corynebacterium glutamicum by transformation or conjugation. After two events of homologous recombination comprising a recombination event within the 5′-flanking sequence provided by the plasmid vector with the homologous sequence of the Corynebacterium glutamicum chromosome and a recombination event within the 3′-flanking sequence provided by the plasmid vector with the homologous sequence of the Corynebacterium glutamicum chromosome, one effecting integration and one effecting excision of said plasmid vector, the mutation is incorporated in the Corynebacterium glutamicum chromosome. Thus the nucleotide sequence of the gene in question contained in the chromosome of said desired strain is replaced by the mutated nucleotide sequence.

An event of homologous recombination may also be referred to as crossing over.

It is preferred that the L-lysine excreting Corynebacterium glutamicum strains of the present invention have the ability to excrete ≥0.25 g/l, preferably ≥0.5 g/l, particularly preferred ≥1,0 g/l of L-lysine in a suitable medium under suitable conditions.

The invention further provides a fermentative process for producing L-lysine, using the Corynebacterium glutamicum of the present invention.

In a fermentative process according to the invention a Corynebacterium glutamicum modified in accordance with the present invention and having the ability to excrete L-lysine is cultivated in a suitable medium under suitable conditions. Due to said ability to excrete said L-lysine the concentration of the L-lysine increases and accumulates in the medium during the fermentative process and the L-lysine is thus produced.

The fermentative process may be discontinuous process like a batch process or a fed batch process or a continuous process. A summary concerning the general nature of fermentation processes is available in the textbook by H. Chmiel (Bioprozesstechnik, Spektrum Akademischer Verlag, 2011), in the textbook of C. Ratledge and B. Kristiansen (Basic Biotechnology, Cambridge University Press, 2006) or in the textbook of V. C. Hass and R. Portner (Praxis der Bioprozesstechnik Spektrum Akademischer Verlag, 2011).

A suitable medium used for the production of L-lysine by a fermentative process contains a carbon source, a nitrogen source, a phosphorus source, inorganic ions and other organic compounds as required.

Suitable carbon sources include glucose, fructose, sucrose as well as the corresponding raw materials like starch hydrolysate, molasses or high fructose corn syrup.

As nitrogen source organic nitrogen-containing compounds such as peptones, meat extract, soy bean hydrolysates or urea, or inorganic compounds such as ammonium sulphate, ammonium chloride, ammonium phosphate, ammonium carbonate, ammonium nitrate, ammonium gas or aqueous ammonia can be used.

As phosphorus source, phosphoric acid, potassium dihydrogen phosphate or dipotassium hydrogen phosphate or the corresponding sodium-containing salts can be used.

Inorganic ions like potassium, sodium, magnesium, calcium, iron and further trace elements etc. are supplied as salts of sulfuric acid, phosphoric acid or hydrochloric acid.

Other organic compounds means essential growth factors like vitamins e g thiamine or biotin or L-amino acids e. g. L-homoserine.

The media components may be added to the culture in form of a single batch or be fed in during the cultivation in a suitable manner.

During the fermentative process the pH of the culture can be controlled by employing basic compounds such as sodium hydroxide, potassium hydroxide, ammonia or aqueous ammonia, or acidic compounds such as phosphoric acid or sulphuric acid in a suitable manner. The pH is generally adjusted to a value of from 6.0 to 8.5, preferably 6.5 to 8.0. To control foaming, it is possible to employ antifoam agents such as, for example, fatty acid polyglycol esters. To maintain the stability of plasmids, it is possible to add to the medium suitable selective substances such as, for example, antibiotics. The fermentative process is preferably carried out under aerobic conditions. In order to maintain these conditions, oxygen or oxygen-containing gas mixtures such as, for example air are introduced into the culture. The fermentative process is carried out, where appropriate, at elevated pressure, for example at an elevated pressure of 0.03 to 0.2 MPa. The temperature of the culture is normally from 25° C. to 40° C., preferably from 30° C. to 37° C. In a discontinuous process, the cultivation is continued until an amount of the L-lysine sufficient for being recovered has been formed. The cultivation is then completed. This aim is normally achieved within 10 hours to 160 hours. In continuous processes, longer cultivation times are possible.

Examples of suitable media and culture conditions can be found inter alia in L. Eggeling and M. Bott (Handbook of Corynebacterium glutamicum, CRC Press, 2005) and the patent documents U.S. Pat. Nos. 5,770,409, 5,990,350, 5,275,940, 5,763,230 and 6,025,169.

Thus the fermentative process results in a fermentation broth which contains the desired L-lysine.

Accordingly a method for the fermentative production of L-lysine is provided comprising the steps of:
a) cultivating a Corynebacterium glutamicum of the present invention in a suitable medium under suitable conditions, and
b) accumulating said L-lysine in the medium to produce an L-lysine containing fermentation broth.

A product containing the L-lysine is then recovered in liquid or solid from the fermentation broth.

A “fermentation broth” means a medium in which a Corynebacterium glutamicum of the invention has been cultivated for a certain time and under certain conditions.

When the fermentative process is completed, the resulting fermentation broth accordingly comprises:
a) the biomass (cell mass) of the Corynebacterium glutamicum of the invention, said biomass having been produced due to propagation of the cells of said Corynebacterium glutamicum,
b) the desired L-lysine accumulated during the fermentative process,
c) the organic by-products accumulated during the fermentative process, and
d) the components of the medium employed which have not been consumed in the fermentative process.

The organic by-products include compounds which may be formed by the Corynebacterium glutamicum of the invention during the fermentative process in addition to production of the L-lysine.

The fermentation broth is removed from the culture vessel or fermentation tank, collected where appropriate, and used for providing a product containing the L-lysine, in liquid or solid form. The expression “recovering the L-lysine-containing product” is also used for this. In the simplest case, the L-lysine-containing fermentation broth itself, which has been removed from the fermentation tank, constitutes the recovered product.

The fermentation broth can subsequently be subjected to one or more of the measures selected from the group consisting of:
a) partial (>0% to <80%) to complete (100%) or virtually complete (≥80%, ≥90%, ≥95%, ≥96%, ≥97%, ≥98%, ≥99%) removal of the water,
b) partial (>0% to <80%) to complete (100%) or virtually complete (≥80%, ≥90%, ≥95%, ≥96%, ≥97%, ≥98%, ≥99%) removal of the biomass, the latter being optionally inactivated before removal,
c) partial (>0% to <80%) to complete (100%) or virtually complete (≥80%, ≥90%, ≥95%, ≥96%, ≥97%, ≥98%, ≥99%, ≥99.3%, ≥99.7%) removal of the organic by-products formed during the fermentative process, and
d) partial (>0% to <80%) to complete (100%) or virtually complete (≥80%, ≥90%, ≥95%, ≥96%, ≥97%, ≥98%, ≥99%, ≥99.3%, ≥99.7%) removal of the residual components of the medium employed or of the residual input materials resp., which have not been consumed in the fermentative process.

An abundance of technical instructions for measures a), b), c) and d) are available in the art.

Removal of water (measure a)) can be achieved inter alia by evaporation, using e.g. a falling film evaporator, by reverse osmosis or nanofiltration. The concentrates thus obtained can be further worked up by spray drying or spray granulation. It is likewise possible to dry the fermentation broth directly using spray drying or spray granulation.

Removal of the biomass (measure b)) can be achieved inter alia by centrifugation, filtration or decantation or a combination thereof.

Removal of the organic by-products (measure c)) or removal of residual components of the medium (measure d) can be achieved inter alia by chromatography, e.g. ion exchange chromatography, treatment with activated carbon or crystallization. In case the organic by-products or residual components of the medium are present in the fermentation broth as solids they can be removed by measure b).

General instructions on separation, purification and granulation methods can be found inter alia in the book of R. Ghosh “Principles of Bioseperation Engineering” (World Scientific Publishing, 2006), the book of F. J. Dechow “Seperation and Purification Techniques in Biotechnology” (Noyes Publications, 1989), the article “Bioseparation” of Shaeiwitz, et al. (Ullmann's Encyclopedia of Industrial Chemistry, Wiley-VCH, 2012) and the book of P. Serno, et al. “Granulieren” (Editio Cantor Verlag, 2007).

A downstream processing scheme for L-lysine products can be found in the article “L-lysine Production” of R. Kelle, et al. (L. Eggeling and M. Bott (Handbook of Corynebacterium glutamicum, CRC Press, 2005)). U.S. Pat. No. 5,279,744 teaches the manufacturing of a purified L-lysine product by ion exchange chromatography. U.S. Pat. No. 5,431,933 teaches the manufacturing of dry L-amino acid products, e.g., an L-lysine product, containing most of the constituents of the fermentation broth.

Thus a concentration or purification of the L-lysine is achieved and a product having the desired content of said L-lysine is provided.

Finally the the L-lysine may be isolated from the culture broth and crystallized in form of a salt, preferably in form of the hydrochloric acid salt of L-lysine.

Analysis of L-lysine to determine its concentration at one or more time(s) during the fermentation can take place by separating the L-lysine by means of ion exchange chromatography, preferably cation exchange chromatography, with subsequent post-column derivatization using ninhydrin, as described in Spackman et al. (Analytical Chemistry 30:1190-1206 (1958)). It is also possible to employ ortho-phthalaldehyde rather than ninhydrin for post-column derivatization. An overview article on ion exchange chromatography can be found in Pickering (LC.GC (Magazine of Chromatographic Science 7(6):484-487 (1989)). It is likewise possible to carry out a pre-column derivatization, for example using ortho-phthalaldehyde or phenyl isothiocyanate, and to fractionate the resulting amino acid derivates by reversed-phase chromatography (RP), preferably in the form of high-performance liquid chromatography (HPLC). A method of this type is described, for example, in Lindroth, et al. (Analytical Chemistry 51:1167-1174 (1979)). Detection is carried out photometrically (absorption, fluorescence). A review regarding amino acid analysis can be found inter alia in the textbook “Bioanalytik” by Lottspeich and Zorbas (Spektrum Akademischer Verlag, Heidelberg, Germany 1998).

### Experimental Section

### A) Materials and Methods

The molecular biology kits, primers and chemicals used and some details of the methods applied are briefly described herewith.
1. Antibiotics and chemicals
   a. Kanamycin: Kanamycin solution from Streptomyces kanamyceticus from Sigma Aldrich (St. Louis, USA, Cat. no. K0254).
   b. Nalidixic acid: Nalidixic acid sodium salt from Sigma Aldrich (St. Louis, USA, Cat. no. N4382).
   c. If not stated otherwise, all chemicals were purchased analytically pure from Merck (Darmstadt, Germany), Sigma Aldrich (St. Louis, USA) or Carl-Roth (Karlsruhe, Germany).
2. Cultivation
   If not stated otherwise, all cultivation/incubation procedures were performed as follows:
   a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Einsbach, Germany) at 37° C. and 200 rpm.
   b. LB agar (MILLER) from Merck (Darmstadt, Germany Cat. no. 110283) was used for cultivation of E. coli strains on agar plates. The agar plates were incubated at 37° C. in an INCU-Line® mini incubator from VWR (Radnor, USA).
   c. Brain heart infusion broth (BHI) from Merck (Darmstadt, Germany; Cat. no. 110493) was used to cultivate C. glutamicum strains in liquid medium. The liquid cultures (10 ml liquid medium per 100 ml Erlenmeyer flask with 3 baffles) were incubated in the Infors HT Multitron standard incubator shaker from Infors GmbH (Einsbach, Germany) at 33° C. and 200 rpm.
   d. Brain heart agar (BHI-agar) from Merck (Darmstadt, Germany; Cat. no. 113825) was used for cultivation of C. glutamicum strains on agar plates. The agar plates were incubated at 33° C. in an incubator from Heraeus Instruments with Kelvitron® temperature controller (Hanau, Germany).
3. Determining optical density
   a. The optical density of bacterial suspensions in shake flask cultures was determined at 600 nm (OD600) using the BioPhotometer from Eppendorf AG (Hamburg, Germany).
   b. The optical density of bacterial suspensions produced in the Wouter Duetz (WDS) micro fermentation system (24-Well Plates) was determined at 660 nm (OD660) with the GENios™ plate reader from Tecan Group AG (Männedorf, Switzerland).
2. Centrifugation
   a. Benchtop centrifuge for reaction tubes with a volume up to 2 ml Bacterial suspensions with a maximum volume of 2 ml were caused to sediment using 1 ml or 2 ml reaction tubes (e.g. Eppendorf Tubes® 3810X) using an Eppendorf 5417 R centrifuge (5 min. at 13.000 rpm).
   b. Benchtop centrifuge for tubes with a volume up to 50 ml
      Bacterial suspensions with a maximum volume of 50 ml were caused to sediment using 15 ml or 50 ml centrifuge tubes (e.g. Falcon™ 50 ml Conical Centrifuge Tubes) using an Eppendorf 5810 R centrifuge for 10 min. at 4.000 rpm.
3. DNA isolation
   a. Plasmid DNA was isolated from E. coli cells using the QIAprep Spin Miniprep Kit from Qiagen (Hilden, Germany, Cat. No. 27106).
   b. Total DNA from C. glutamicum was isolated using the method of Eikmanns et al. (Microbiology 140, 1817-1828, 1994).
4. Polymerase chain reaction (PCR)
   PCR with a proof reading (high fidelity) polymerase was used to amplify a desired segment of DNA before assembly or Sanger sequencing.
   a. The Phusion® High-Fidelity DNA Polymerase Kit (NEB Phusion Kit) from New England BioLabs Inc. (Ipswich, USA; Cat. No. M0530) was used for template-correct amplification of selected DNA regions according to the instructions of the manufacturer (see table 1).

TABLE 1
Thermocycling conditions for PCR with Phusion ®
High-Fidelity DNA Polymerase Kit from NEB Inc.
PCR-program

| | Time | T | |
|---|---|---|---|
| Step | [min.] | [° C.] | Description |
| 1 | 00:30 | 98 | Initial denaturation step |
| 2 | 00:05 | 98 | Denaturation step |
| 3 | 00:30 | 60 | Annealing step |
| 4 | 00:xx | 72 | Elongation step |
| | | | 15-30 sec. per kb DNA |
| | | | Repeat step 2 to 4: 30x |
| 5 | 05:00 | 72 | Final Elongation step |
| 6 | Hold | 4 | Cooling step |

b. Primer
   The oligonucleotides used were synthesized by eurofins genomics GmbH (Ebersberg, Germany) using the phosphoramidite method described by McBride and Caruthers (Tetrahedron Lett. 24:245-248, 1983).
c. Template
   As PCR template either a suitably diluted solution of isolated plasmid DNA or of isolated total DNA from a C. glutamicum liquid culture or the total DNA contained in a colony was used (colony PCR). For said colony PCR the template was prepared by taking cell material with a toothpick from a colony on an agar plate and placing the cell material directly into the PCR reaction tube. The cell material was heated for 10 sec. with 800 W in a microwave oven type Mikrowave & Grill from SEVERIN Elektrogerate GmbH (Sundern, Germany) and then the PCR reagents were added to the template in the PCR reaction tube.
d. PCR Cycler
PCR's were carried out in PCR Cyclers type Mastercycler or Mastercycler nexus gradient from Eppendorf AG (Hamburg, Germany).
5. Detection of mutations using FRET

The presence of a given mutation, e.g., a nucleobase exchange, was detected by real-time PCR in combination with FRET hybridization probes. The term FRET is the abbreviation for fluorescence resonance energy transfer. As real-time PCR instrument a Lightcycler from Roche Diagnostics® was used (see below).

This method was, e.g., used by M. J. Lay and C. T. Wittwer (Clinical Chemistry 42 (12):2262-2267 (1997)) for the genotyping of factor V Leiden. Cyril DS Mamotte (The Clinical Biochemist Reviews 27:63-75 (2006)) reviews the genotyping of single nucleotide substitutions using this method. Summaries concerning this method may be found in the textbooks Lewin's Genes XII by Jocelyn E. Krebs, Elliott S. Goldstein and Stephan T. Kilpatrick (Jones and Bartlett Publishers, US, 2018), Molecular Diagnostics, 12 Tests that changed everything by W. Edward Highsmith (Humana Press, Springer, New York, 2014) or elsewhere in the art.

The FRET hybridization donor probe was labelled with the fluorescent dye fluorescein and the acceptor probe with the fluorescent dye LC-Red640. In essence the detection method comprised three steps: colony PCR, probe hybridization and subsequent melting curve analysis. The method is simply referred to as real-time PCR herewith.
a. Primers and Probes
   The oligonucleotides used were synthesized by eurofins genomics GmbH (Ebersberg, Germany).
b. Template
   As PCR template the total DNA contained in a colony was used. It was prepared by taking cell material with a toothpick from a colony on an agar plate and placing the cell material directly into the PCR reaction tube. The cell material was heated for 10 sec. with 800 W in a microwave oven type Mikrowave & Grill from SEVERIN Elektrogerate GmbH (Sundern, Germany) and then the PCR reagents were added to the template in the PCR reaction tube.
c. Reaction Mix
   The Type-it® Fast SNP probe PCR Kit (Type-it Kit) from Qiagen (Hilden, Germany, Cat.No. 206045) was used for real-time detection of the mutations. Therefore 2.5 μl of the Qiagen Fast SNP Puffer (2×) was mixed with 0.5 μl of each of the LC-PCR-Primers [10 μM] and 0.5 μl of each of the 1:500 diluted acceptor and donor probe [100 pmol/μl] to get the mastermix for the real-time PCR.

TABLE 2
Thermocycling conditions for PCR with
the LightCycler ® (step 1-3) and
melting curve analysis (step 4-6).
PCR-program

| | Time | T | |
|---|---|---|---|
| Step | [sec.] | [° C.] | Description |
| 1 | 15 | 95 | Denaturation step |
| | | | (and Activation of |
| | | | HotStarTaq ™ DNA |
| | | | polymerase) |
| 2 | 05 | 55 | Annealing step |
| 3 | 30 | 72 | Elongation step |
| | | | Repeat step 1 to 3: |
| | | | 50x |
| 4 | 10 | 95 | Denaturation step |
| 5 | 30 | 40 | Probe hybridisation |
| 6 | | 40-80 | Melting curve |
| | | | analysis |
| 7 | | 80-40 | Cooling |

d. PCR Cycler

The reactions were carried out in a LightCycler® 2.0 Instrument and analysed with LightCycler® Software 4.1 of Roche Diagnostics (Rotkreuz, Switzerland).
6. Restriction enzyme digestion of DNA

The FastDigest restriction endonucleases (FD) and the associated buffer from ThermoFisher Scientific (Waltham, USA) were used for restriction digestion of plasmid DNA. The reactions were carried out according to the instructions of the manufacturer's manual
7. Determining the size of DNA fragments

The size of DNA fragments was determined by automatic capillary electrophoresis using the QIAxcel from Qiagen (Hilden, Germany).
8. Purification of PCR amplificates and restriction fragments

PCR amplificates and restriction fragments were cleaned up using the QIAquick PCR Purification Kit from Qiagen (Hilden, Germany; Cat. No. 28106), according to the manufacturer's instructions.
9. Determining DNA concentration

DNA concentration was measured using the NanoDrop Spectrophotometer ND-1000 from PEQLAB Biotechnologie GmbH, since 2015 VWR brand (Erlangen, Germany).
10. Gibson Assembly

Vectors allowing integration of the desired mutation into the chromosome were made using the method of Gibson, et al. (Science 319:1215-20, 2008). The Gibson Assembly Kit from New England BioLabs Inc. (Ipswich, USA; Cat. No. E2611) was used for this purpose. The reaction mix, containing the restricted vector and at least one DNA insert, was incubated at 50° C. for 60 min. 0.5 μl of the Assembly mixture was used for a transformation experiment.
11. Chemical transformation of E. coli
   a. Chemically competent E. coli Stellar™ cells were purchased from Clontech Laboratories Inc. (Mountain View, USA; Cat. No. 636763) and transformed according to the manufacturer's protocol (PT5055-2).
      These cells were used as transformation hosts for reaction mixtures obtained by Gibson Assembly. The transformation batches were cultivated overnight for approximately 18 h at 37° C. and the transformants containing plasmids selected on LB agar supplemented with 50 mg/l kanamycin.
   b. E. coli K-12 strain S17-1 was used as donor for conju-gational transfer of plasmids based on pK18mobsacB from E. coli to C. glutamicum. Strain S17-1 is described by Simon, R., et al. (Bio/Technology 1:784-794, 1983). It is available from the American Type Culture Collection under the access number ATCC47055.
      Chemically competent E. coli S17-1 cells were made as follows: A preculture of 10 ml LB medium (10 ml liquid medium per 100 ml Erlenmeyer flask with 3 baffles) was inoculated with 100 μl bacterial suspension of strain S17-1 and the culture was incubated overnight for about 18 h at 37° C. and 250 rpm. The main culture (70 ml LB contained in a 250 ml Erlenmeyer flask with 3 baffles) was inoculated with 300 μl of the preculture and incubated up to an OD600 of 0.5-0.8 at 37° C. The culture was centrifuged for 6 min. at 4° C. and 4000 rpm and the supernatant was discarded. The cell pellet was resuspended in 20 ml sterile, ice-cold 50 mM CaCl₂ solution and incubated on ice for 30 min. After another centrifugation step, the pellet was resuspended in 5 ml ice-cold 50 mM CaCl₂ solution and the suspension incubated on ice for 30 min. The cell suspension was then adjusted to a final concentration of 20% glycerol (v/v) with 85% (v/v) sterile ice-cold glycerol. The suspension was divided into 50 μl aliquots and stored at −80° C.
      To transform S17-1 cells, the protocol according to Tang, et al. (Nucleic Acids Res. 22(14):2857-2858, 1994) with a heat shock of 45 sec. was used.
12. Conjugation of C. glutamicum

The pK18mobsacB plasmid system described by Schafer et al. (Gene 145, 69-73, 1994) was used to integrate desired DNA fragments into the chromosome of C. glutamicum. A modified conjugation method of Schafer et al. (Journal of Bacteriology 172, 1663-1666, 1990) was used to transfer the respective plasmid into the desired C. glutamicum recipient strain. Liquid cultures of the C. glutamicum strains were carried out in BHI medium at 33° C. The heat shock was carried out at 48.5° C. for 9 min. Transconjugants were selected by plating the conjugation batch on EM8 agar (Table 4), which was supplemented with 25 mg/l kanamycin and 50 mg/l nalidixic acid. The EM8 agar plates were incubated for 72 h at 33° C.

TABLE 4
Composition of the EM8 agar

| | Concentration |
|---|---|
| Components | (g/l) |
| Glucose (sterile-filtered) | 23 |
| CSL (corn steep liquor) | 30 |
| Peptone from soymeal (Merck, Germany) | 40 |
| (NH₄)₂SO₄ | 8 |
| Urea | 3 |
| KH₂PO₄ | 4 |
| MgSO₄•7 H₂O | 0.5 |
| FeSO₄•7 H₂O | 0.01 |
| CuSO₄•5 H₂O | 0.001 |
| ZnSO₄•7 H₂O | 0.01 |
| Calcium pantothenate, D(+) | 0.01 |
| Thiamine | 0.001 |
| Inositol | 0.1 |
| Nicotinic acid | 0.001 |
| Biotin (sterile-filtered) | 0.005 |
| CaCO₃ (autoclaved separately) | 1.6 |
| Agar-Agar (Merck, Germany) | 14 |

Sterile toothpicks were used to transfer the transconjugants onto BHI agar, which was supplemented with 25 mg/l kanamycin and 50 mg/l nalidixic acid. The agar plates were incubated for 20 h at 33° C. The cultures of the respective transconjugants produced in this manner were then propagated further for 24 h at 33° C. in 10 ml BHI medium contained in 100 ml Erlenmeyer flasks with 3 baffles. An aliquot was taken from the liquid culture suitably diluted and plated (typically 100 to 200 μl) on BHI agar which was supplemented with 10% saccharose. The agar plates were incubated for 48 h at 33° C. The colonies growing on the saccharose containing agar plates were then examined for the phenotype kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined for integration of the desired DNA fragment by means of real-time PCR.
13. Determining nucleotide sequences

Nucleotide sequences of DNA molecules were determined by eurofins genomics GmbH (Ebersberg, Germany) by cycle sequencing, using the dideoxy chain termination method of Sanger, et al. (Proceedings of the National Academy of Sciences USA 74:5463-5467, 1977), on Applied Biosystems® (Carlsbad, Calif., USA) 3730x1 DNA Analyzers. Clonemanager Professional 9 software from Scientific & Educational Software (Denver, USA) was used to visualise and evaluate the sequences.
14. Glycerol stocks of E. coli and C. glutamicum strains

For long time storage of E. coli- and C. glutamicum strains glycerol stocks were prepared. Selected E. coli clones were cultivated in 10 ml LB medium supplemented with 2 g/l glucose. Selected C. glutamicum clones were cultivated in two fold concentrated BHI medium supplemented with 2 g/l glucose. Cultures of plasmid containing E. coli strains were supplemented with 50 mg/l kanamycin. Cultures of plasmid containing C. glutamicum strains were supplemented with 25 mg/l kanamycin. The medium was contained in 100 ml Erlenmeyer flasks with 3 baffles. It was inoculated with a loop of cells taken from a colony and the culture incubated for about 18 h at 37° C. and 200 rpm in the case of E. coli and 33° C. and 200 rpm in the case of C. glutamicum. After said incubation period 1.2 ml 85% (v/v) sterile glycerol were added to the culture. The obtained glycerol containing cell suspension was then aliquoted in 2 ml portions and stored at −80° C.
15. Cultivation system according to Wouter Duetz (WDS)

The milliliter-scale cultivation system according to Duetz (Trends Microbiol. 2007; 15(10):469-75) was used to investigate the performance of the C. glutamicum strains constructed. For this purpose, 24-deepwell microplates (24 well WDS plates) from EnzyScreen BV (Heemstede, Netherlands; Cat. no. CR1424), filled with 2.5 mL medium were used.

Precultures of the strains were done in 10 ml two fold concentrated BHI medium. The medium was contained in a 100 ml Erlenmeyer flask with 3 baffles. It was inoculated with 100 μl of a glycerol stock culture and the culture incubated for 24 h at 33° C. and 200 rpm.

After said incubation period the optical densities OD600 of the precultures were determined. The main cultures were done by inoculating the 2.5 ml medium containing wells of the 24 Well WDS-Plate with an aliquot of the preculture to give an optical density OD600 of 0.1. As medium for the main culture CGXII medium described by Keilhauer, et al. (J. Bacteriol. 175(17): 5595-5603 Sept. 1993) was used. For convenience the composition of the CGXII medium is shown in table 8.

TABLE 8
Composition of Keilhauer's CGXII medium.

| Components | Concentration (g/l) |
|---|---|
| MOPS (3-(N-Morpholino)propanesulfonic acid) | 42 |
| (NH₄)₂SO₄ | 20 |
| Urea | 5 |
| KH₂PO₄ | 1 |
| K₂HPO₄ | 1 |
| MgSO₄•7 H₂O | 0.25 |
| CaCl₂ | 0.01 |
| FeSO₄•7 H₂O | 0.01 |
| MnSO₄ H₂O | 0.01 |
| ZnSO₄•7 H₂O | 0.001 |
| CuSO₄•5 H₂O | 0.0002 |
| NiCl₂ 6H₂O | 0.00002 |
| Biotin (sterile-filtered) | 0.0002 |
| Protocatechuic acid (sterile-filtered) | 0.03 |
| Carbon source (sterile-filtered) | as needed |
| adjust the pH to 7 with NaOH | |

These main cultures were incubated for approximately 45 h at 33° C. and 300 rpm in an Infors HT Multitron standard incubator shaker from Infors GmbH (Bottmingen, Switzerland) until complete consumption of glucose.

The glucose concentration in the suspension was analysed with the blood glucose-meter OneTouch Vita® from LifeScan (Johnson & Johnson Medical GmbH, Neuss, Germany).

After cultivation the culture suspensions were transferred to a deep well microplate. A part of the culture suspension was suitably diluted to measure the OD600. Another part of the culture was centrifuged and the concentration of L-amino acids, in particular L-lysine, and residual glucose were analysed in the supernatant.
16. Amino acid analyser

The concentration of L-lysine and other L-amino acids, e.g. L-valine, in the culture supernatants was determined by ion exchange chromatography using a SYKAM 5433 amino acid analyser from SYKAM Vertriebs GmbH (Fürstenfeldbruck, Germany). As solid phase a column with spherical, polystyrene-based cation exchanger (Peek LCA N04/Na, dimension 150×4 6 mm) from SYKAM was used. Depending on the L-amino acid the separation takes place in an isocratic run using a mixture of buffers A and B for elution or by gradient elution using said buffers. As buffer A an aquous solution containing in 20 1 263 g trisodium citrate, 120 g citric acid, 1100 ml methanol, 100 ml 37% HCl and 2 ml octanoic acid (final pH 3.5) was used. As buffer B an aquous solution containing in 20 1 392 g trisodium citrate, 100 g boric acidand 2 ml octanoic acid (final pH 10.2) was used. The free amino acids were coloured with ninhydrin through post-column derivatization and detected photometrically at 570 nm.
17. Glucose determination with continuous flow system (CFS)

A SANplus multi-channel continuous flow analyser from SKALAR analytic GmbH (Erkelenz, Germany) was used to determine the concentration of glucose in the supernatant. Glucose was detected with a coupled-enzyme assay (Hexokinase/Glucose-6-Phosphate-Dehydrogenase) via NADH formation.

### B) Experimental Results

### Example 1

Sequence of the Mqo Gene of C. glutamicum Strain DM1933 and DM2463

Strain DM1933 is an L-lysine producer described by Blombach, et al. (Applied and Environmental Microbiology 75(2):419-427, 2009). It is deposited according to the Budapest treaty at the DSMZ under accession number DSM25442.

Strain DM2463 is an L-lysine producer obtained from ATCC13032 during a multi step strain breeding program aiming at increased performance parameters for L-lysine production. The nucleotide sequence of the chromosome of strain DM1933 and of strain DM2463 was determined by Illumina whole-genome sequencing technology (Illumina Inc., San Diego, Calif., US). See e.g., Benjak, et al. (2015) Whole-Genome Sequencing for Comparative Genomics and De Novo Genome Assembly. In: Parish T., Roberts D. (eds) Mycobacteria Protocols. Methods in Molecular Biology, Vol 1285. Humana Press, NY, US) and Bennet, S. (Pharmacogenomics 5(4):433-438, 2004).

It was found that the nucleotide sequence of the mqo coding sequence of strain DM1933 including the nucleotide sequence upstream and downstream thereof is identical to that of ATCC13032 shown in SEQ ID NO:1.

It was found that the nucleotide sequence of the mqo coding sequence of strain DM2463 including the sequence of 1000 nucleobases upstream and 1003 nucleobases downstream thereof is different in two positions to that of ATCC13032 shown in SEQ ID NO:1. Instead of nucleobase thymine at position 1683 of SEQ ID NO:1 the corresponding nucleobase in strain DM2463 is adenine. Instead of nucleobase cytosine at position 2230 of SEQ ID NO:1 the corresponding nucleobase in strain DM2463 is thymine.

As a consequence of the mutation at position 1683 the amino acid at position 228 of the encoded amino acid sequence of the Mqo polypeptide of strain DM2463 is aspartic acid (abbreviated as D or Asp). The amino acid at position 228 of the encoded amino acid sequence of the Mqo polypeptide of strain ATCC13032 is valine (abbreviated as V or Val) as shown in SEQ ID NO:2. This alteration or exchange resp. in the amino acid sequence from valine to aspartic acid is abbreviated as V228D.

The mutation at position 2230 is a silent mutation. It does not lead to an alteration of the amino acid at position 410 of the encoded amino acid sequence, which is phenylalanine. The nucleotide sequence of the mqo coding sequence of strain DM2463 including the nucleotide sequence upstream and downstream thereof is also shown in SEQ ID NO:3. The amino acid sequence of the encoded Mqo polypeptide of strain DM2463 is shown in SEQ ID NO:4. DM1933 contains in its chromosome a variant of the aspartokinase gene encoding a feed back resistant aspartokinase polypeptide. Said feed back resistant aspartokinase polypeptide has the amino acid sequence of SEQ ID NO:6 of the sequence listing, wherein the amino acid threonine (Thr) at position 311 of the amino acid sequence is replaced by isoleucine (Ile). In U.S. Pat. No. 7,338,790 the abbreviation “lysC T311I” is used to indicate said exchange. Blombach et al. use the abbreviation “lysC(T311I)”.

### Example 2

Construction of Plasmid pK18mobsacB_Mqo′_V228D

Plasmid pK18mobsacB_mqo′_V228D was constructed to enable incorporation of the mutation causing the amino acid exchange V228D (see example 1) into the nucleotide sequence of the mqo coding sequence of strain DM1933. The plasmid is based on the mobilizable vector pK18mobsacB described by Schafer, et al. (Gene 145 69-73, 1994). For the construction of pK18mobsacB_mqo′_V228D the Gibson Assembly method was used.

For this purpose two polynucleotides or DNA molecules resp. were made: One polynucleotide called mqo′_V228D comprised the 5′-end of the mqo coding sequence including the mutation leading to the amino acid exchange of valine at position 228 to aspartate (V228D). The second polynucleotide was plasmid pK18mobsacB linearized by treatment with restriction endonuclease XbaI.

Polynucleotide mqo′_V228D was synthesized by PCR using total DNA isolated from a C. glutamicum DM2463 culture as template and oligonucleotides 1f-mqo-pK18 and 1r-mqo-pK18 as primers (table 9). The primers are also shown in SEQ ID NO:9 and SEQ ID NO:10 of the sequence listing. For PCR the Phusion Kit was used with an elongation step (see table 4, step 4) of 30 sec.

TABLE 9
List of primers used and size of amplific ate during Phusion Kit PCR.

| synthesis | | | |
|---|---|---|---|
| of amplificate | name | sequence | size [bp] |
| mqo′_V228D | 1f-mqo-pK18 | GGTACCCGGGGATCCTCCCACGCGTCAGTC | 1634 |
| | | AAAA (SEQ ID NO: 9) | |
| | 1r-mqo-pK18 | GCCTGCAGGTCGACTAGGGTCTTCTGGGTG | |
| | | CGT (SEQ ID NO: 10) | |

The nucleotide sequence of the amplificate mqo′_V228D is shown in SEQ ID NO:11. It contains the nucleotide sequence shown in SEQ ID NO:7.

Amplificate mqo′_V228D contains a sequence of 801 bps length upstream and a sequence of 801 bps downstream from the mutation (see position 818 of SEQ ID NO:11 or position 802 of SEQ ID NO:7) causing the amino acid exchange from valine (V) to aspartic acid (D) in strain DM2463. At its 5′-end and 3′-end it is equipped with a nucleotide sequence each overlapping with the corresponding sequence of pK18mobsacB cut with XbaI.

Said overlapping sequences are required for the Gibson assembly technique.

Plasmid pK18mobsacB was linearized with the restriction endonuclease XbaI. The digestion mixture was subsequently controlled by capillary electrophoresis, purified and the DNA concentration quantified.

To assemble the plasmid pK18mobsacB_mqo′_V228D the two polynucleotides i.e. the vector pK18mobsacB cut with XbaI and the amplificate mqo′_V228D were mixed with the Gibson Assembly Kit ingredients. The assembly mixture thus obtained was used to transform chemically competent E. coli Stellar™ cells.

22 kanamycin resistant transformants were analyzed by real-time PCR (see table 2) using the Type-it Kit and the primers LC-mqo1 and LC-mqo2 for PCR amplification and oligonucleotides mqo228_C as acceptor probe and mqo228_A as donor probe for melting curve analysis. The primers and probes are shown in table 10 and under SEQ ID NO:13 to SEQ ID NO:16 of the sequence listing.

TABLE 10
List of primers and probes used for real-time PCR.

| name | sequence |
|---|---|
| LC-mqo1 | ATGAAGGCACCGACATCAAC (SEQ ID NO: 13) |
| LC-mqo2 | TGCAAGGCGATTAAGTTGGG (SEQ ID NO: 14) |
| mqo228_C¹ | ACGTTCTTGTCGGTCACGAT (SEQ ID NO: 15) |
| mqo228_A² | GAAGTTTGCCTTGATGGTCTTGGTGTCGCCAGTGT |
| | (SEQ ID NO: 16) |

¹acceptor probe labelled with LC-Red640 at the 5′-end and phosphorylated at the 3′-end
²donor probe labelled with fluorescein at the 3′-end

Plasmid DNA from three transformants thus characterized as containing the desired mutation was isolated and the polynucleotide mqo′_V228D created within the plasmid during the Gibson assembly analyzed by Sanger sequencing. For sequence analysis the primers pVW_1.p, pCV22_2. p (table 11), LC-mqo1 and LC-mqo2 (table 10) were used. They are also shown under SEQ ID NO:17 and 18 and SEQ ID NO:13 and 14 of the sequence listing.

TABLE 11
List of primers used for Sanger sequencing.

| analysis of | name | sequence |
|---|---|---|
| mqo′_V228D | pVW_1.p | GTGAGCGGATAACAATTTCACAC |
| | | (SEQ ID NO: 17) |
| | pCV22_2.p | TGCAAGGCGATTAAGTTGGG |
| | | (SEQ ID NO: 18) |

The analysis of the nucleotide sequences thus obtained showed that the polynucleotide mqo′_V228D contained in the plasmid of the three transformants analyzed had the nucleotide sequence presented in SEQ ID NO:11. One of said transformants was called Stellar/pK18mobsacB_mqo′_V228D and saved as a glycerol stock for further experiments.

### Example 3

Construction of Strain DM1933_mqo_V228D

The plasmid pK18mobsacB_mqo′_V228D obtained in example 2 was used to incorporate the mutation (see position 818 of SEQ ID NO:11) leading to the amino acid exchange V228D (see SEQ ID NO:12) into the chromosome of the L-lysine producer DM1933.

Chemically competent cells of E. coli strain S17-1 were transformed with plasmid DNA of pK18mobsacB_mqo′_V228D. The modified conjugation method of Schafer et al. (Journal of Bacteriology 172, 1663-1666, 1990) as described in materials and methods was used for conjugal transfer into the strain DM1933 and for selection of transconjugant clones by virtue of their saccharose resistance and kanamycin sensitivity phenotype.

Transconjugant clones were analyzed by real-time PCR using the Type-it Kit and the primers LC-mqo1 and LC-mqo2 for PCR amplification and mqo228_C as acceptor probe and mqo228_A as donor probe for melting curve analysis (table 10).

One of the transconjugant clones thus characterized was called DM1933_mqo_V228D. A glycerol stock culture of the transconjugant clone was prepared and used as starting material for further investigations.

The nucleotide sequence of the chromosomal region of strain DM1933_mqo_V228D containing the mutated nucleotide sequence was analyzed by Sanger sequencing.

For this purpose a PCR amplificate was produced spanning the site of mutation. A colony PCR was done using the primers mqo-E1 and mqo-oP1 (see table 12) and the NEB Phusion Kit with an elongation time of 45 sec. (table 1, step 4). The amplificate obtained was then sequenced using the primers LC-mqo1 and LC-mqo2 (see table 10). The nucleotide sequences of the primers used in this context are also shown in SEQ ID NO:19 and 20.

TABLE 12
List of primers used for colony PCR.

| amplification/ | | | size |
|---|---|---|---|
| detection of | name | sequence | [bp] |
| mqo_V228D | mqo-E1 | GCCTTCAACTGTGTCAGTTC (SEQ ID NO: 19) | 1648 |
| | mqo-oP1 | GAGGATCCGCAGAGAACTCGCGGAGATA | |
| | | (SEQ ID NO: 20) | |

The nucleotide sequence obtained is shown in SEQ ID NO:21. The result showed that strain DM1933_mqo_V228D contained the desired mutation, or the desired mutated nucleotide sequence resp., in its chromosome.

Thus the mqo gene of strain DM1933 was mutated with the effect that the amino acid valine at position 228 of the amino acid sequence of the encoded Mqo polypeptide was replaced by aspartic acid.

### Example 4

L-Lysine Production by Strain DM1933_mqo_V228D

Strains DM1933 (reference) and DM1933_mqo_V228D obtained in example 3 were analyzed for their ability to produce L-lysine from glucose by batch cultivation using the cultivation system according to Wouter Duetz.

As medium CGXII containing 20 g/l glucose as carbon source was used. The cultures were incubated for 45 h until complete consumption of glucose as confirmed by glucose analysis using blood glucose-meter and the concentrations of L-lysine and the optical density OD660 were determined. The result of the experiment is presented in table 14.

TABLE 14
L-lysine production by strain DM1933_mqo_V228D.

| strain | L-lysine¹ (g/l) | OD660 |
|---|---|---|
| DM1933 | 3.9 | 8.3 |
| DM1933_mqo_V228D | 4.3 | 8.0 |

¹as L-lysine × HCl

The experiment shows that L-lysine production was increased in strain DM1933_mqo_V228D as compared to the parent strain DM1933.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

What is claimed is:

1. A bacterium of the species Corynebacterium glutamicum having the ability to excrete L-lysine, comprising in its chromosome: a polynucleotide encoding a polypeptide having the activity of a malate:quinone oxidoreductase and comprising the amino acid sequence of SEQ ID NO:2, wherein the amino acid valine at position 228 is substituted by a different proteinogenic amino acid.

2. The bacterium of claim 1, wherein said amino acid at position 228 of the amino acid sequence of SEQ ID NO:2 is aspartic acid or glutamic acid.

3. The bacterium of claim 1, wherein said amino acid at position 228 of the amino acid sequence of SEQ ID NO:2 is aspartic acid.

4. The bacterium of claim 3, wherein the polynucleotide encoding said amino acid sequence comprises the nucleotide sequence of positions 1001 to 2500 of SEQ ID NO:1 and wherein the nucleobase at position 1683 is adenine.

5. The bacterium of claim 3, wherein the polynucleotide encoding said amino acid sequence comprises the nucleotide sequence of positions 1001 to 2500 of SEQ ID NO:3.

6. The bacterium of claim 1, wherein the 5′-end of the polynucleotide encoding said amino acid sequence is directly connected to the 3′-end of the polynucleotide comprising the nucleotide sequence from positions 781 to 1000 of SEQ ID NO:1 or of SEQ ID NO:3.

7. The bacterium of claim 6, wherein the polynucleotide encoding said amino acid sequence comprises the nucleotide sequence from positions 781 to 2500 of SEQ ID NO:3.

8. The bacterium of claim 6, wherein the polynucleotide encoding said amino acid sequence comprises the nucleotide sequence from positions 781 to 2500 of SEQ ID NO:1 and wherein the nucleobase at position 1683 being adenine.

9. A method for the fermentative production of L-lysine comprising the steps of:
a) cultivating the bacterium of claim 1 in a suitable medium under suitable conditions, and
b) accumulating L-lysine in the medium to form an L-lysine containing fermentation broth.

10. The method of claim 9, wherein said method comprises manufacturing an L-lysine containing product from said fermentation broth.

11. The method of claim 9, wherein said method comprises extracting or substantially eliminating water from said fermentation broth.

12. The method of claim 11, wherein at least 40% (w/w) of the water is extracted from the fermentation broth.

13. The method of claim 11, wherein at least 90% (w/w) of the water is extracted from the fermentation broth.

14. The method of claim 11, wherein at least 95% (w/w) of the water is extracted from the fermentation broth.

15. The method of claim 12, wherein said manufacturing comprises a purification step.

16. The method of claim 15, wherein said purification step is selected from the group consisting of: treatment with charcoal; ionic exchange; and crystallization.

17. The method of claim 10, further comprising isolation of L-lysine from the fermentation broth and crystallization of L-lysine in the form of a salt.

18. The bacterium of claim 2, wherein the 5′-end of the polynucleotide encoding said amino acid sequence is directly connected to the 3′-end of the polynucleotide comprising the nucleotide sequence from positions 781 to 1000 of SEQ ID NO:1 or of SEQ ID NO:3.

19. The bacterium of claim 18, wherein the polynucleotide encoding said amino acid sequence comprises the nucleotide sequence from positions 781 to 2500 of SEQ ID NO:3.

20. The bacterium of claim 18, wherein the polynucleotide encoding said amino acid sequence comprises the nucleotide sequence from positions 781 to 2500 of SEQ ID NO:1 and wherein the nucleobase at position 1683 being adenine.


SEQUENCE LISTING (22 sequences)

SEQ ID NO: 1 - DNA, 3503 bp, Corynebacterium glutamicum ATCC13032
  misc_feature (1)..(3503): nucleotide sequence comprising locus_tag NCgl1926 disclosed in GenBank accession number NC_003450
  misc_feature (781)..(781): nucleobase adenine
  CDS (1001)..(2500)
  misc_feature (1682)..(1684): gtc codon for valine at position 228
  misc_feature (1683)..(1683): nucleobase thymine
  misc_feature (2228)..(2230): ttc codon for phenylalanine at position 410
  misc_feature (2230)..(2230): nucleobase cytosine
  misc_feature (2501)..(2503): stop codon
tccgtatgctcccaaacctccgcctgggatgatggttgtgggggagcgtttgcccaacaccctcaccataggggtgatcagcttgatgaacagtggtggatacatcatgtccagccacggactgttgaggaccagcgctggaatcttctcaatggcagctggattgcttgtgcgcatctgggacatccacagaggaacgatgagtccaccagtggaatgggcgacggggactagctcagggtgggtggaagagatgacctcggcagcagctgttaagtcagggaagtaatgggcaagatcagaggtgtagtgccactgctgtcctggacggtaggagcgtccacattttctaagatcaatgccgtacacagcaaaaccggcattgtggaaaaactccgcgaattcagtgtggaagaagtagtccgtcatgccgtgaacccacagcaacgctgggcgggcagcaaaagactcgtccgcgtgattgtctgggttgtagcgcacaaccgttgtcacaacatctgtttcattatccgggtcgtcgccgagctcgatggtgaggttttggtagccctctcccaggaagtctggtttccactgctgcatctgattcatacgtataacgataggtcgattgttggtgtggtgtgcgcgagtcgactgaaatgttcacgtggtgaaacttccgcgatactactcatgtttgcgaattgcacatttactaactttgcaaattgggggagggggtagcgcgggggaggaattcgcatgagaaaggggaatatcccgtgcttgtttattcagctcgaggtggcaggcgtacactctatattcacggacaatgtgtacccacgctttcttgtaagaaacaagaagggtaacgccccacgcgtcagtcaaaaatatggccaacacttgcattcgggtgctggcgatcatttatgagatgacgccttgtgttggtgttcggcagagaactcgcggagataaaaggaagttgaacatgtcagattccccgaagaacgcaccgaggattaccgatgaggcagatgtagttctcattggtgccggtatcatgagctccacgctgggtgcaatgctgcgtcagctggagccaagctggactcagatcgtcttcgagcgtttggatggaccggcacaagagtcgtcctccccgtggaacaatgcaggaaccggccactctgctctatgcgagctgaactacaccccagaggttaagggcaaggttgaaattgccaaggctgtaggaatcaacgagaagttccaggtttcccgtcagttctggtctcacctcgttgaagagggagtgctgtctgatcctaaggaattcatcaaccctgttcctcacgtatctttcggccagggcgcagatcaggttgcatacatcaaggctcgctacgaagctttgaaggatcacccactcttccagggcatgacctacgctgacgatgaagctaccttcaccgagaagctgcctttgatggcaaagggccgtgacttctctgatccagtagcaatctcttggatcgatgaaggcaccgacatcaactacggtgctcagaccaagcagtacctggatgcagctgaagttgaaggcactgaaatccgctatggccacgaagtcaagagcatcaaggctgatggcgcaaagtggatcgtgaccgtcaagaacgtacacactggcgacaccaagaccatcaaggcaaacttcgtgttcgtcggcgcaggcggatacgcactggatctgcttcgcagcgcaggcatcccacaggtcaagggcttcgctggattcccagtatccggcctgtggcttcgttgcaccaacgaggaactgatcgagcagcacgcagccaaggtatatggcaaggcatctgttggcgctcctccaatgtctgttcctcaccttgacacccgcgttatcgagggtgaaaagggtctgctctttggaccttacggtggctggacccctaagttcttgaaggaaggctcctacctggacctgttcaagtccatccgcccagacaacattccttcctaccttggcgttgctgctcaggaatttgatctgaccaagtaccttgtcactgaagttctcaaggaccaggacaagcgtatggatgctcttcgcgagtacatgccagaggcacaaaacggcgattgggagaccatcgttgccggacagcgtgttcaggttattaagcctgcaggattccctaagttcggttccctggaattcggcaccaccttgatcaacaactccgaaggcaccatcgccggattgctcggtgcttcccctggagcatccatcgcaccttccgcaatgatcgagctgcttgagcgttgcttcggtgaccgcatgatcgagtggggcgacaagctgaaggacatgatcccttcctacggcaagaagcttgcttccgagccagcactgtttgagcagcagtgggcacgcacccagaagaccctgaagcttgaggaagcctaaatcttctaactgctttctttaaagcacccgcacatgtctgttgaggtttcacctgcggagacaatctccgccttcatgggttggaactgacacagttgaaggcatgtcgggtgctttgcgtattctttgccagtgtgatttaggcgacaccaataatttattgggtatccaccaattaccgctgtgagcactgcaaattacgtattcgaaaagccatgtccaccacgtgttctatcctggcggcatgcaaaaaatcaccccaaacatctggtgccaaggcaccgcagacgaagcagccgaattctacgtcaatgcgttttctgagtttccgggtggcgcagaagtactcaccacagttaagtatcccgaagctggcttgctggacttccaggagcctttcgcaggaaaaaccttgacggtggaactcgctatctcaggctttaagatcatcttgatcaatgctggtgaagagttcactcccaacccatcgatcagcttcatggtgaattttgatgcggtgcgtgatgaaaatgccaaagagcaccttgatgcggtgtgggaaaaactccatgaaggcggcagcacactgatgccagtcgatacttacccattttcggaatactacgggtgggtacaagacaaatatggtgtgagctggcaattgatgctcagccgcccagaagaaaagccaggtcccgcagtaatcccaacgctcttatttggtggggcagctcaaaatcaggcaggcccagctcaagaaaactacgttgaggtgttcccgaactcccaacttggtgatcgtgcaccttatggacagcaaacaggtcctgccactcctgaggccctcatgttttcccagttccaactcgacggtcagtggattttcgcgatggattccggagttgagcaagatttcaccttcagtgagggtgtctcattgatgtatgaagctcatggtcaagaagaactcgatgccatctggaatgcactctcggcagtt

SEQ ID NO: 2 - protein, 500 aa, Corynebacterium glutamicum ATCC13032
MSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTVKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKTLKLEEA

SEQ ID NO: 3 - DNA, 3503 bp, Corynebacterium glutamicum
  misc_feature (781)..(781): nucleobase adenine
  CDS (1001)..(2500)
  misc_feature (1682)..(1684): gac codon for aspartic acid at position 228
  mutation (1683)..(1683): nucleobase adenine
  misc_feature (2228)..(2230): ttt codon for phenylalanine at position 410
  misc_feature (2230)..(2230): nucleobase thymine
  misc_feature (2501)..(2503): stop codon
tccgtatgctcccaaacctccgcctgggatgatggttgtgggggagcgtttgcccaacaccctcaccataggggtgatcagcttgatgaacagtggtggatacatcatgtccagccacggactgttgaggaccagcgctggaatcttctcaatggcagctggattgcttgtgcgcatctgggacatccacagaggaacgatgagtccaccagtggaatgggcgacggggactagctcagggtgggtggaagagatgacctcggcagcagctgttaagtcagggaagtaatgggcaagatcagaggtgtagtgccactgctgtcctggacggtaggagcgtccacattttctaagatcaatgccgtacacagcaaaaccggcattgtggaaaaactccgcgaattcagtgtggaagaagtagtccgtcatgccgtgaacccacagcaacgctgggcgggcagcaaaagactcgtccgcgtgattgtctgggttgtagcgcacaaccgttgtcacaacatctgtttcattatccgggtcgtcgccgagctcgatggtgaggttttggtagccctctcccaggaagtctggtttccactgctgcatctgattcatacgtataacgataggtcgattgttggtgtggtgtgcgcgagtcgactgaaatgttcacgtggtgaaacttccgcgatactactcatgtttgcgaattgcacatttactaactttgcaaattgggggagggggtagcgcgggggaggaattcgcatgagaaaggggaatatcccgtgcttgtttattcagctcgaggtggcaggcgtacactctatattcacggacaatgtgtacccacgctttcttgtaagaaacaagaagggtaacgccccacgcgtcagtcaaaaatatggccaacacttgcattcgggtgctggcgatcatttatgagatgacgccttgtgttggtgttcggcagagaactcgcggagataaaaggaagttgaacatgtcagattccccgaagaacgcaccgaggattaccgatgaggcagatgtagttctcattggtgccggtatcatgagctccacgctgggtgcaatgctgcgtcagctggagccaagctggactcagatcgtcttcgagcgtttggatggaccggcacaagagtcgtcctccccgtggaacaatgcaggaaccggccactctgctctatgcgagctgaactacaccccagaggttaagggcaaggttgaaattgccaaggctgtaggaatcaacgagaagttccaggtttcccgtcagttctggtctcacctcgttgaagagggagtgctgtctgatcctaaggaattcatcaaccctgttcctcacgtatctttcggccagggcgcagatcaggttgcatacatcaaggctcgctacgaagctttgaaggatcacccactcttccagggcatgacctacgctgacgatgaagctaccttcaccgagaagctgcctttgatggcaaagggccgtgacttctctgatccagtagcaatctcttggatcgatgaaggcaccgacatcaactacggtgctcagaccaagcagtacctggatgcagctgaagttgaaggcactgaaatccgctatggccacgaagtcaagagcatcaaggctgatggcgcaaagtggatcgtgaccgacaagaacgtacacactggcgacaccaagaccatcaaggcaaacttcgtgttcgtcggcgcaggcggatacgcactggatctgcttcgcagcgcaggcatcccacaggtcaagggcttcgctggattcccagtatccggcctgtggcttcgttgcaccaacgaggaactgatcgagcagcacgcagccaaggtatatggcaaggcatctgttggcgctcctccaatgtctgttcctcaccttgacacccgcgttatcgagggtgaaaagggtctgctctttggaccttacggtggctggacccctaagttcttgaaggaaggctcctacctggacctgttcaagtccatccgcccagacaacattccttcctaccttggcgttgctgctcaggaatttgatctgaccaagtaccttgtcactgaagttctcaaggaccaggacaagcgtatggatgctcttcgcgagtacatgccagaggcacaaaacggcgattgggagaccatcgttgccggacagcgtgttcaggttattaagcctgcaggatttcctaagttcggttccctggaattcggcaccaccttgatcaacaactccgaaggcaccatcgccggattgctcggtgcttcccctggagcatccatcgcaccttccgcaatgatcgagctgcttgagcgttgcttcggtgaccgcatgatcgagtggggcgacaagctgaaggacatgatcccttcctacggcaagaagcttgcttccgagccagcactgtttgagcagcagtgggcacgcacccagaagaccctgaagcttgaggaagcctaaatcttctaactgctttctttaaagcacccgcacatgtctgttgaggtttcacctgcggagacaatctccgccttcatgggttggaactgacacagttgaaggcatgtcgggtgctttgcgtattctttgccagtgtgatttaggcgacaccaataatttattgggtatccaccaattaccgctgtgagcactgcaaattacgtattcgaaaagccatgtccaccacgtgttctatcctggcggcatgcaaaaaatcaccccaaacatctggtgccaaggcaccgcagacgaagcagccgaattctacgtcaatgcgttttctgagtttccgggtggcgcagaagtactcaccacagttaagtatcccgaagctggcttgctggacttccaggagcctttcgcaggaaaaaccttgacggtggaactcgctatctcaggctttaagatcatcttgatcaatgctggtgaagagttcactcccaacccatcgatcagcttcatggtgaattttgatgcggtgcgtgatgaaaatgccaaagagcaccttgatgcggtgtgggaaaaactccatgaaggcggcagcacactgatgccagtcgatacttacccattttcggaatactacgggtgggtacaagacaaatatggtgtgagctggcaattgatgctcagccgcccagaagaaaagccaggtcccgcagtaatcccaacgctcttatttggtggggcagctcaaaatcaggcaggcccagctcaagaaaactacgttgaggtgttcccgaactcccaacttggtgatcgtgcaccttatggacagcaaacaggtcctgccactcctgaggccctcatgttttcccagttccaactcgacggtcagtggattttcgcgatggattccggagttgagcaagatttcaccttcagtgagggtgtctcattgatgtatgaagctcatggtcaagaagaactcgatgccatctggaatgcactctcggcagtt

SEQ ID NO: 4 - protein, 500 aa, Corynebacterium glutamicum
MSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTDKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKTLKLEEA

SEQ ID NO: 5 - DNA, 1266 bp, Corynebacterium glutamicum ATCC13032
  CDS (1)..(1263)
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaa

SEQ ID NO: 6 - protein, 421 aa, Corynebacterium glutamicum ATCC13032
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 7 - DNA, 1603 bp, Corynebacterium glutamicum
  misc_feature (1)..(801): sequence upstream of the site of mutation (5′-flanking sequence)
  CDS (120)..(1601)
  misc_feature (801)..(803): gac codon for aspartic acid at position 228
  mutation (802)..(802): nucleobase adenine
  misc_feature (803)..(1603): sequence downstream of the site of mutation (3′-flanking sequence)
  misc_feature (1347)..(1349): ttt codon for phenyalanine at position 410
  misc_feature (1349)..(1349): nucleobase thymine
cccacgcgtcagtcaaaaatatggccaacacttgcattcgggtgctggcgatcatttatgagatgacgccttgtgttggtgttcggcagagaactcgcggagataaaaggaagttgaacatgtcagattccccgaagaacgcaccgaggattaccgatgaggcagatgtagttctcattggtgccggtatcatgagctccacgctgggtgcaatgctgcgtcagctggagccaagctggactcagatcgtcttcgagcgtttggatggaccggcacaagagtcgtcctccccgtggaacaatgcaggaaccggccactctgctctatgcgagctgaactacaccccagaggttaagggcaaggttgaaattgccaaggctgtaggaatcaacgagaagttccaggtttcccgtcagttctggtctcacctcgttgaagagggagtgctgtctgatcctaaggaattcatcaaccctgttcctcacgtatctttcggccagggcgcagatcaggttgcatacatcaaggctcgctacgaagctttgaaggatcacccactcttccagggcatgacctacgctgacgatgaagctaccttcaccgagaagctgcctttgatggcaaagggccgtgacttctctgatccagtagcaatctcttggatcgatgaaggcaccgacatcaactacggtgctcagaccaagcagtacctggatgcagctgaagttgaaggcactgaaatccgctatggccacgaagtcaagagcatcaaggctgatggcgcaaagtggatcgtgaccgacaagaacgtacacactggcgacaccaagaccatcaaggcaaacttcgtgttcgtcggcgcaggcggatacgcactggatctgcttcgcagcgcaggcatcccacaggtcaagggcttcgctggattcccagtatccggcctgtggcttcgttgcaccaacgaggaactgatcgagcagcacgcagccaaggtatatggcaaggcatctgttggcgctcctccaatgtctgttcctcaccttgacacccgcgttatcgagggtgaaaagggtctgctctttggaccttacggtggctggacccctaagttcttgaaggaaggctcctacctggacctgttcaagtccatccgcccagacaacattccttcctaccttggcgttgctgctcaggaatttgatctgaccaagtaccttgtcactgaagttctcaaggaccaggacaagcgtatggatgctcttcgcgagtacatgccagaggcacaaaacggcgattgggagaccatcgttgccggacagcgtgttcaggttattaagcctgcaggatttcctaagttcggttccctggaattcggcaccaccttgatcaacaactccgaaggcaccatcgccggattgctcggtgcttcccctggagcatccatcgcaccttccgcaatgatcgagctgcttgagcgttgcttcggtgaccgcatgatcgagtggggcgacaagctgaaggacatgatcccttcctacggcaagaagcttgcttccgagccagcactgtttgagcagcagtgggcacgcacccagaagaccct

SEQ ID NO: 8 - protein, 494 aa, Corynebacterium glutamicum
MSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTDKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKT

SEQ ID NO: 9 - DNA, 34 bp, artificial sequence
  primer
  misc_feature (1)..(34): primer 1f-mqo-pK18
ggtacccggggatcctcccacgcgtcagtcaaaa

SEQ ID NO: 10 - DNA, 33 bp, artificial sequence
  primer
  misc_feature (1)..(33): primer 1r-mqo-pK18
gcctgcaggtcgactagggtcttctgggtgcgt

SEQ ID NO: 11 - DNA, 1634 bp, artificial sequence
  amplificate from Corynebacterium glutamicum DM2463
  misc_feature (1)..(1634): polynucleotide mqo′_V228D
  misc_feature (1)..(16): 5′-overlap to pK18mobsacB-XbaI
  misc_feature (17)..(1619): nucleotide sequence of SEQ ID NO7
  misc_feature (17)..(1619): nucleotide sequence of DM2463
  misc_feature (17)..(817): sequence upstream of the site mutation (5′-flanking sequence)
  CDS (136)..(1617)
  misc_feature (817)..(819): gac codon for aspartic acid at position 228
  mutation (818)..(818): nucleobase adenine
  misc_feature (819)..(1619): sequence downstream of the site of mutation (3′-flanking sequence)
  misc_feature (1363)..(1365): ttt codon for phenylalanine at position 410
  misc_feature (1365)..(1365): nucleobase thymine
  misc_feature (1620)..(1634): 3′-overlap to pK18mobsacB-XbaI
ggtacccggggatcctcccacgcgtcagtcaaaaatatggccaacacttgcattcgggtgctggcgatcatttatgagatgacgccttgtgttggtgttcggcagagaactcgcggagataaaaggaagttgaacatgtcagattccccgaagaacgcaccgaggattaccgatgaggcagatgtagttctcattggtgccggtatcatgagctccacgctgggtgcaatgctgcgtcagctggagccaagctggactcagatcgtcttcgagcgtttggatggaccggcacaagagtcgtcctccccgtggaacaatgcaggaaccggccactctgctctatgcgagctgaactacaccccagaggttaagggcaaggttgaaattgccaaggctgtaggaatcaacgagaagttccaggtttcccgtcagttctggtctcacctcgttgaagagggagtgctgtctgatcctaaggaattcatcaaccctgttcctcacgtatctttcggccagggcgcagatcaggttgcatacatcaaggctcgctacgaagctttgaaggatcacccactcttccagggcatgacctacgctgacgatgaagctaccttcaccgagaagctgcctttgatggcaaagggccgtgacttctctgatccagtagcaatctcttggatcgatgaaggcaccgacatcaactacggtgctcagaccaagcagtacctggatgcagctgaagttgaaggcactgaaatccgctatggccacgaagtcaagagcatcaaggctgatggcgcaaagtggatcgtgaccgacaagaacgtacacactggcgacaccaagaccatcaaggcaaacttcgtgttcgtcggcgcaggcggatacgcactggatctgcttcgcagcgcaggcatcccacaggtcaagggcttcgctggattcccagtatccggcctgtggcttcgttgcaccaacgaggaactgatcgagcagcacgcagccaaggtatatggcaaggcatctgttggcgctcctccaatgtctgttcctcaccttgacacccgcgttatcgagggtgaaaagggtctgctctttggaccttacggtggctggacccctaagttcttgaaggaaggctcctacctggacctgttcaagtccatccgcccagacaacattccttcctaccttggcgttgctgctcaggaatttgatctgaccaagtaccttgtcactgaagttctcaaggaccaggacaagcgtatggatgctcttcgcgagtacatgccagaggcacaaaacggcgattgggagaccatcgttgccggacagcgtgttcaggttattaagcctgcaggatttcctaagttcggttccctggaattcggcaccaccttgatcaacaactccgaaggcaccatcgccggattgctcggtgcttcccctggagcatccatcgcaccttccgcaatgatcgagctgcttgagcgttgcttcggtgaccgcatgatcgagtggggcgacaagctgaaggacatgatcccttcctacggcaagaagcttgcttccgagccagcactgtttgagcagcagtgggcacgcacccagaagaccctagtcgacctgcaggc

SEQ ID NO: 12 - protein, 494 aa, artificial sequence; identical to SEQ ID NO: 8
  Synthetic Construct

SEQ ID NO: 13 - DNA, 20 bp, artificial sequence
  primer
  misc_feature (1)..(20): primer LC-mqo1
atgaaggcaccgacatcaac

SEQ ID NO: 14 - DNA, 20 bp, artificial sequence
  primer
  misc_feature (1)..(20): primer LC-mqo2
tgcaaggcgattaagttggg

SEQ ID NO: 15 - DNA, 20 bp, artificial sequence
  probe
  misc_feature (1)..(20): acceptor probe mqo228_C
  misc_feature (10)..(10): nucleobase thymine
acgttcttgtcggtcacgat

SEQ ID NO: 16 - DNA, 35 bp, artificial sequence
  probe
  misc_feature (1)..(35): donor probe mqo228_A
gaagtttgccttgatggtcttggtgtcgccagtgt

SEQ ID NO: 17 - DNA, 23 bp, artificial sequence
  primer
  misc_feature (1)..(23): primer pVW_1.p
gtgagcggataacaatttcacac

SEQ ID NO: 18 - DNA, 20 bp, artificial sequence
  primer
  misc_feature (1)..(20): primer pCV22_2.p
tgcaaggcgattaagttggg

SEQ ID NO: 19 - DNA, 20 bp, artificial sequence
  primer
  misc_feature (1)..(20): primer mqo-E1
gccttcaactgtgtcagttc

SEQ ID NO: 20 - DNA, 28 bp, artificial sequence
  primer
  misc_feature (1)..(28): primer mqo-oP1
gaggatccgcagagaactcgcggagata

SEQ ID NO: 21 - DNA, 1648 bp, artificial sequence
  amplificate from Corynebacterium glutamicum strain DM1933_mqo_V228D
  misc_feature (1)..(28): nucleotide sequence of primer mqo-oP1
  misc_feature (9)..(1648): nucleotide sequence from strain DM1933_mqo_V228D
  CDS (43)..(1542)
  misc_feature (724)..(726): gac codon for aspartic acid at position 228
  misc_feature (725)..(725): nucleobase adenine
  misc_feature (1270)..(1272): ttt codon for phenylalanine at position 410
  misc_feature (1271)..(1271): nuclebase thymine
  misc_feature (1543)..(1545): stop codon
  misc_feature (1630)..(1648): nucleotide sequence of the reverse complement of primer mqo-E1
gaggatccgcagagaactcgcggagataaaaggaagttgaacatgtcagattccccgaagaacgcaccgaggattaccgatgaggcagatgtagttctcattggtgccggtatcatgagctccacgctgggtgcaatgctgcgtcagctggagccaagctggactcagatcgtcttcgagcgtttggatggaccggcacaagagtcgtcctccccgtggaacaatgcaggaaccggccactctgctctatgcgagctgaactacaccccagaggttaagggcaaggttgaaattgccaaggctgtaggaatcaacgagaagttccaggtttcccgtcagttctggtctcacctcgttgaagagggagtgctgtctgatcctaaggaattcatcaaccctgttcctcacgtatctttcggccagggcgcagatcaggttgcatacatcaaggctcgctacgaagctttgaaggatcacccactcttccagggcatgacctacgctgacgatgaagctaccttcaccgagaagctgcctttgatggcaaagggccgtgacttctctgatccagtagcaatctcttggatcgatgaaggcaccgacatcaactacggtgctcagaccaagcagtacctggatgcagctgaagttgaaggcactgaaatccgctatggccacgaagtcaagagcatcaaggctgatggcgcaaagtggatcgtgaccgacaagaacgtacacactggcgacaccaagaccatcaaggcaaacttcgtgttcgtcggcgcaggcggatacgcactggatctgcttcgcagcgcaggcatcccacaggtcaagggcttcgctggattcccagtatccggcctgtggcttcgttgcaccaacgaggaactgatcgagcagcacgcagccaaggtatatggcaaggcatctgttggcgctcctccaatgtctgttcctcaccttgacacccgcgttatcgagggtgaaaagggtctgctctttggaccttacggtggctggacccctaagttcttgaaggaaggctcctacctggacctgttcaagtccatccgcccagacaacattccttcctaccttggcgttgctgctcaggaatttgatctgaccaagtaccttgtcactgaagttctcaaggaccaggacaagcgtatggatgctcttcgcgagtacatgccagaggcacaaaacggcgattgggagaccatcgttgccggacagcgtgttcaggttattaagcctgcaggatttcctaagttcggttccctggaattcggcaccaccttgatcaacaactccgaaggcaccatcgccggattgctcggtgcttcccctggagcatccatcgcaccttccgcaatgatcgagctgcttgagcgttgcttcggtgaccgcatgatcgagtggggcgacaagctgaaggacatgatcccttcctacggcaagaagcttgcttccgagccagcactgtttgagcagcagtgggcacgcacccagaagaccctgaagcttgaggaagcctaaatcttctaactgctttctttaaagcacccgcacatgtctgttgaggtttcacctgcggagacaatctccgccttcatgggttggaactgacacagttgaaggc

SEQ ID NO: 22 - protein, 500 aa, artificial sequence; identical to SEQ ID NO: 4
  Synthetic Construct